(19) United States
(12) Patent Application Publication
(10) Pub. No.: US 2012/0078356 A1
(43) Pub. Date: Mar. 29, 2012

(54) PERCUTANEOUSLY DELIVERABLE HEART OR BLOOD VESSEL VALVE WITH FRAME HAVING ABLUMINALLY SITUATED TISSUE MEMBRANE

(75) Inventors: R. David FISH, Houston, TX (US); David PANIAGUA, Houston, TX (US); Eduardo INDUNI, Alajuela (CR)

(73) Assignee: COLIBRI HEART VALVE LLC, Broomfield, CO (US)

(21) Appl. No.: 13/243,980

(22) Filed: Sep. 23, 2011

Related U.S. Application Data
(60) Provisional application No. 61/385,867, filed on Sep. 23, 2010.

(51) Int. Cl. A61F 2/24; B21D 51/16

(52) U.S. Cl. 623/2.17; 29/890.12

(57) ABSTRACT

A prosthetic valve implantable by catheter without surgery includes a frame with an abluminal surface extending between a proximal end of the frame and a distal end of the frame, and a single layer of a biocompatible membrane material mounted to the abluminal surface of the frame. The single layer of biocompatible membrane is located such that an interior surface of the membrane sheet extends between the proximal end of the frame and the distal end of the frame, and resides radially exterior the abluminal surface of the frame. In at least one embodiment, the disposition of membrane sheet at all points of attachment is entirely exterior/abluminal to the frame, such that no part of the abluminal surface of the membrane sheet contacts the frame.

### CROSS-REFERENCE TO RELATED APPLICATIONS

The present application claims the benefit of U.S. Provisional Patent Application No. 61/385,867 filed on Sep. 23, 2010, the content of which is incorporated herein by reference in its entirety. Cross-reference is made to U.S. patent application Ser. No. 13/038,361 filed on Mar. 1, 2011, the content of which is incorporated herein by reference.

### FIELD

The present invention relates to the field of medical devices, and more particularly, to a percutaneously deliverable heart valve and to a percutaneously deliverable blood vessel valve.

### BACKGROUND

Heart valve disease is a common degenerative condition that compromises physiologic function and causes limiting symptoms and threat to life in millions of patients all over the world. There are various underlying causes, but malfunction of heart valves is ultimately expressed as insufficient conduction of blood through the plane of the valve due to narrowing of the anatomic pathway (stenosis), or as incompetent closure that allows blood to return back through the valve again, thereby reducing the effective forward conduction of blood through the valve (insufficiency or regurgitation). These hemodynamic states lead to 1) deficiency of cardiac output and 2) adverse loads on the pumping chambers of the heart, both of which in turn lead to functional compromise of the patient and often premature death unless effectively corrected.

Definitive corrective treatment of heart valve disease is conventionally performed by open-chest surgical techniques, wherein the valve is manipulated, repaired, or replaced with a prosthetic valve under direct vision. Heart valve surgery is performed in hundreds of thousands of cases yearly world-wide, but carries a high burden of cost, morbidity, and mortality, especially in susceptible patients who may be elderly or otherwise physiologically compromised by collateral disease. Further, the costs and resource requirements of the surgical enterprise restrict the availability of heart valve replacement to many more patients all over the world.

In pursuit of alternatives to heart valve surgery, over the last ten years a number of development programs have brought percutaneous, trans-catheter implantation of prosthetic heart valves into commercial use in the European Union (EU) and into pivotal clinical trials in the United States of America. Initial clinical experience in the EU was directed toward patients who had critical aortic valve stenosis, but were deemed to be at unacceptably high risk for open-heart surgical valve replacement. In several thousand such cases, utilizing both balloon-expandable and self-expanding designs in two separate programs, percutaneous heart valve replacement (PHVR) was shown to be feasible and possibly competitive with surgery in selected patients with 12-18 month mortality rates of about 25%. Grube E., et al., Progress and Current Status of Percutaneous Aortic Valve Replacement: Results of Three Device Generations of the CoreValve Revalving System, Circ. Cardiovasc Intervent. 2008;1:167-175.

Typically, the current percutaneous heart valve (PHV) designs, including the commercialized Medtronic CoreValve and the Edwards Lifesciences Sapien valves, comprise a biological membrane forming the operating leaflets of the valve, mounted within the interior of a metal frame, that is then collapsed onto a delivery catheter or balloon, and then constrained within an outer sheath. After an initial dilation of the diseased valve with a large balloon, this assembly is then advanced to the plane of the valve and deployed by self-expansion or by balloon expansion.

PHV designs are confronted by several central challenges. More particularly, the functioning valve leaflets are typically constructed of flexible and compressible tissue membrane valve members attached by sutures to a surrounding stent frame that together must be durable, yet of sufficiently low mass to allow for passage in collapsed form into the patient's body through an anatomic pathway—a peripheral artery, for example—of limited diameter, leading to the implantation site within the central circulation system. This condition favors simple, yet robust design geometries.

Secondly, the PHV in its implanted operating configuration must emulate both the opening mechanics and the closing mechanics of the native heart valve—two differing geometries and mechanical forms afforded by the native anatomy of the aortic valve, for example, but with the limitation that the PHV must effectively embody both within its physical and operational envelope without the benefit of the grossly different anatomical forms native to the aortic valve.

As a practical matter, the measures of effective function are simple—the pressure gradient during forward passage of blood across the valve must be as low as possible, typically 5-10 mmHg or less. While achieving this, the “success” of operation in the closed configuration, wherein the leaflets are pressed together along lines of apposition by the pressure of the blood pumped beyond the valve, would also appear to be simply measured by the amount of retrograde blood passage back into the pumping chamber—the “regurgitation” or “leakage.”

However, since this closed phase of valve function is the phase in which the principal force loads are applied to the valve membrane leaflets, and since the manner in which the design of the valve distributes these forces determines the durability of the valve, the real measure of the valve's closing function is best understood by how well the design minimizes and distributes the force loads on the valve leaflets. To date, this problem has not been sufficiently addressed.

In the field of blood vessel diseases certain conditions may be advantageously treated by insertion of valves into an affected patient's blood vessels. Currently no such valve devices are available, though investigation of this approach has suggested potential clinical utility for blood vessel valves, and in particular for valves to be inserted into the vein system for particular conditions. In the first example, insufficiency of the inlet (atrioventricular) tricuspid valve to the right ventricle of the heart results in regurgitation of blood back into the right atrium, which, serving to receive blood flow returning in the veins from the entire body, then results in turn in suffusion and swelling (edema) of all the organs, most notably in the abdomen and extremities, insufficient forward conduction of blood flow from the right ventricle into the lungs causing compromise of pulmonary function, and ultimately pump failure of the right heart. Collectively these conditions are termed right heart failure, a condition that leads to incapacity and possibly to death if progressive and uncorrected. Often, the remedy is surgical repair or replacement of the tricuspid valve, but results are uncertain, damage to the right ventricle being often irreversible, and progressive heart failure may supervene despite technically successful valve surgery.

In a yet a further example, insufficiency of vein function due to the incompetence or destruction of intrinsic valves within the vein system leads to acute then chronic swelling of the veins and their dependent lymphatics and tissues. This condition can affect the deep veins of the body, commonly the lower extremities or pelvis, or the superficial veins of the lower extremities in particular, leading to progressive expansion of the veins and further valvular incompetence, a condition known as varicose veins. Millions of people worldwide suffer from these conditions and enormous funds are expended on procedures to destroy or remove these dilated incompetent veins. It has long been hoped that some form of implantable valve for the vein system could alleviate these conditions.

Several references of interest have been reviewed in preparation of the present disclosure. The applicants do not admit that the any one or more of the following references constitute citable prior art.

U.S. Pat. No. 7,758,632 to Hojeibane discloses a valve construct wherein all embodiments include stent portions that act as proximal and distal anchors that are interconnected by connecting members, and further include a “cantilever valve strut” that acts as a biasing arm to “facilitate the opening and closing of the membrane assembly.” Such structures may disrupt the flow channel and potentially interfere with membrane integrity when crimping the valve to mount it on an expandable balloon. In addition, at the point of engagement of the tissue against the connecting members, there is relatively intense focal stress along the straight connecting member—especially at the free edge of the leaflet. Hojeibane further utilizes flaps 403 and cusps 404 that may be independent components attached to the tubular membrane to form the membrane assembly 102. Accordingly, Hojeibane does not appear to use a flat sheet of membrane.

U.S. Pat. No. 7,025,780 to Gabbay discloses two separate uses of a device referred to as a “stent.” The first use is that of the stent in a surgical valve wherein it is a supportive structure to give shape and mechanical support to the tissue leaflets formed upon it. This device in Gabbay is like a surgical tissue valve. As shown in FIGS. 5 and 6 of Gabbay, the stent is disposed outside of at least an inner tissue leaflet layer. In the second use, as shown in FIGS. 1 and 2 of Gabbay, a tissue valve of some type is disposed within an outer frame of the vascular stent type. In this case, the tissue layer is not disposed upon the abluminal surface of the outer stent frame. The reader is directed to column 1, lines 61-63 of Gabbay that state “The prosthesis includes a valve apparatus located within a stent apparatus to form a stented valve.” Gabbay further references only a “valve apparatus comprising an animal pulmonic heart valve.” Accordingly, Gabbay fails to disclose a valve formed of flat tissue membrane wherein the tissue membrane is attached to the abluminal surface of a frame.

U.S. Patent Application Publication No. 2006/0190074 to Hill is directed to venous valves, and as such, the structural embodiments shown in Hill do not appear robust enough for application as prosthetic heart valves, such as in the aortic valve position. The valve material is referred to as a “cover” comprising a matrix and “integrated flexible support members 124”—essentially a reinforcing layer applied to the matrix. While tissue sources of “extracellular membrane” are cited as possible sources for the matrix, the use of a single layer tissue membrane for the leaflets is not disclosed in Hill.

With further reference to U.S. Patent Application Publication No. 2006/0190074, Hill also does not describe how the cover material is attached to the frame to achieve a sufficiently robust construct for utilization as a prosthetic heart valve. That is, while Hill generally discusses attachment of the cover to the frame at Paragraph [0072] using a variety of possible fasteners, none are shown and described relative to the frame. Of particular relevance is that while Hill mentions coupling the cover 108 to the frame 102 at connection regions 132 and 134, there is no mention of coupling the cover 108 to the arcuate portions of the frame members 126 that lead to the connection regions 132 and 134.

Accordingly, there is a need to address the shortcomings discussed above.

### SUMMARY

It is to be understood that the present invention includes a variety of different versions or embodiments, and this Summary is not meant to be limiting or all-inclusive. This Summary provides some general descriptions of some of the embodiments, but may also include some more specific descriptions of other embodiments.

As noted above, the real measure of the valve's closing function is best understood by how well the design minimizes and distributes the force loads on the valve leaflets. This condition favors design geometries in which closing apposition of the leaflet surfaces is achieved with a minimum of traction force on the valve attachment points to the frame. To this end the inventive valve achieves this and other operational advantages by situating the operating tissue membrane to the exterior/abluminal surface of the valve frame rather than the interior/luminal space of the frame and by distributing the operating force loads of the valve along the curved edges forming the distal (downstream to flow direction) end of the frame. No other known percutaneously implantable or even surgical valve bioprosthesis utilizes this configuration with the tissue membrane mounted entirely upon the abluminal aspect of the device frame which carries the closed valve force loads along the distal formed edge of the frame corresponding to the lines of attachment of the leaflet membrane.

Accordingly, in at least one embodiment, an implantable prosthetic valve is provided that includes a frame and tissue membrane. Advantageously, the tissue membrane resides to the exterior of the frame along an axial length of the frame in the flow direction of the implantable prosthetic valve when implanted. That is, the membrane sheet resides entirely exterior or abluminal to the frame when the valve is in the fully open condition and at least at all attachment points when the valve is partly or completely closed. The attachment points may comprise a plurality of sutures that are used to attach the membrane sheet to the frame at a variety of locations, such as at one or more intersections of the frame.

The descriptions of the inventive valve are focused for the purpose of technical specification upon the replacement heart valve application, but will apply as well to the blood vessel valve device. By way of example, in addition to use of the valves described herein to replace heart valves, methods and devices described herein also provide for transcatheter implantation of a valve into the inferior vena cava (the principal conduit vein from the lower body inserting into the right heart) to act as an upstream substitute in part for the tricuspid valve. Such a valve device would be advantageously designed to be low in mass with large effective orifice. The inventive valve device is proposed as suitable to this purpose. Alternatively, the condition of right heart failure may be treated in part by interposing valves into the vein system farther upstream in the venous return flow, such as in the subclavian or principal iliac veins.

Accordingly, in at least one embodiment, an implantable prosthetic valve is provided for controlling, at least in part, a flow of blood, comprising:

a frame having an abluminal frame surface, a proximal end, and a distal end, wherein the proximal end is situated at an inlet end of the frame relative to the flow of blood when implanted, and wherein the distal end is situated at an outlet end of the frame relative to the flow of blood when implanted, the frame having a tubular flow path through its interior; and

a tissue membrane attached to the frame, the tissue membrane having an interior surface and an exterior surface;

wherein the interior surface of the tissue membrane is situated exterior the abluminal frame surface of the frame between the proximal end and distal end of the frame, when the valve is in the fully open position, the interior surface of the tissue membrane intersecting the tubular flow path of the frame when the tissue membrane is located in a closed position.

A percutaneous, trans-catheter prosthetic valve for implantation in a patient is provided, comprising:

a frame including an abluminal surface extending between a proximal end of the frame and a distal end of the frame, wherein the frame is collapsible and expandable and adapted for trans-catheter delivery; and

a biocompatible tissue material mounted to the abluminal surface of the frame to form a plurality of valve leaflets, wherein an entire interior surface of the biocompatible tissue material between the proximal end of the frame and the distal end of the frame resides radially exterior to the abluminal surface of the frame:
(a) at all points of attachment; and
(b) when the plurality of valve leaflets are in an operationally fully open position.

In at least one embodiment the frame comprises a metal alloy substantially configured as tubular stent member. In at least one embodiment a proximal portion of the frame includes a ring. In at least one embodiment a proximal portion of the frame comprises a circumferential zig-zag of wire. In at least one embodiment a proximal portion of the frame includes a lattice. In at least one embodiment the lattice is circumferentially continuous. In at least one embodiment the lattice is circumferentially discontinuous. In at least one embodiment a distal end of the frame includes two or more areas of axial continuity with the proximal end, wherein the two or more areas of axial continuity comprise axially oriented projections. In at least one embodiment the frame further comprises a distally positioned stabilization framework comprising at least one of circumferential or radial continuity with the axially oriented projections. In at least one embodiment the frame includes two or more regions of circumferential discontinuity through which operating leaflets of the biocompatible tissue material move radially inward and outward in closing and opening operation, respectively. In at least one embodiment the biocompatible tissue material between the proximal end of the frame and the distal end of the frame resides substantially adjacent the abluminal surface of the frame. In at least one embodiment the biocompatible tissue material does not contact a luminal surface of the frame. In at least one embodiment an exterior surface of the biocompatible tissue material does not contact a luminal surface of the frame.

In accordance with at least one embodiment, the frame can be a closed cell lattice type construct of circumferentially corrugated/sinusoidal/zig-zag rings. In accordance with at least one embodiment, the frame can be a wire loop with axial loops forming a support for each commissure. In at least one embodiment, the frame includes a proximal portion, wherein at least some of the abluminal surface of the proximal portion includes a tissue sheet attached thereto.

In at least one embodiment, a prosthetic valve for implantation in a patient is provided, comprising:

a frame including an abluminal surface extending between a proximal edge of the frame and a distal edge of the frame, the distal edge undulating axially to define at least two areas of circumferential discontinuity in the frame, wherein the frame is collapsible and expandable and adapted for trans-catheter delivery; and

a single layer of a biocompatible membrane material mounted to the abluminal surface of the frame to form leaflet portions, wherein the leaflet portions are collocated with the at least two areas of circumferential discontinuity in the frame.

In at least one embodiment the leaflet portions are attached to the frame at least along curved frame members formed by the distal edge of the frame and corresponding to the radially outward boundaries of the leaflet cusps.

In at least one embodiment, no portion of the biocompatible membrane material is mounted to an interior surface of the frame. In at least one embodiment, the frame comprises a metal alloy substantially configured as tubular stent member. In at least one embodiment, a proximal portion of the frame includes a lattice to which the biocompatible membrane material is circumferentially mounted entirely upon the abluminal aspect of the tubular stent member. In at least one embodiment, at least some proximal portion of the frame does not include biocompatible membrane material mounted to its luminal or abluminal surfaces. In at least one embodiment, the biocompatible membrane material extends between the proximal edge and the distal edge of the frame. In at least one embodiment, a distal portion of the frame further includes a distally extending stabilizing framework comprising a plurality of axially oriented support members that each extend from a distally extending frame projection situated adjacent the at least two areas of circumferential discontinuity in the frame. In at least one embodiment, the prosthetic valve further comprises a plurality of radial support members interconnecting the axially oriented support members. In at least one embodiment, the prosthetic valve further comprises a wire guide, wherein the wire guide is coaxially aligned with an axis of the valve, and wherein the wire guide is configured to allow for a coaxial passage of a guide wire such that coaxial alignment of the distally extending stabilizing framework may be facilitated during valve deployment. In at least one embodiment, the wire guide comprises at least one of a ring and a tube.

A method of preparing a percutaneous, trans-catheter prosthetic valve is also provided, the method comprising mounting a single layer of a biocompatible tissue material to an abluminal surface of a trans-catheter deliverable frame such that an interior surface of the biocompatible tissue material between a proximal end of the trans-catheter deliverable frame and a distal end of the trans-catheter deliverable frame resides radially exterior to and substantially adjacent the abluminal surface of the trans-catheter deliverable frame. In at least one embodiment the method further comprises compressing and crimping the trans-catheter deliverable frame, with the biocompatible tissue material mounted thereto, upon a delivery catheter. In at least one embodiment the method further comprises implanting the trans-catheter deliverable frame with the biocompatible tissue material mounted thereto into a patient. In at least one embodiment the trans-catheter deliverable frame comprises a stent. In at least one embodiment the method further comprises mounting the trans-catheter deliverable frame and the biocompatible tissue material mounted thereto on a mandrel.

In accordance with at least one embodiment, a method of constructing a prosthetic valve is provided, the method, comprising attaching a biocompatible membrane material to a collapsible and expandable frame to form a trans-catheter deliverable prosthetic valve, wherein an entire interior surface of the biocompatible membrane material is located exterior of the abluminal surface of the collapsible and expandable frame when leaflet portions of the biocompatible membrane material are in the valve's operationally open position. In at least one embodiment, the method further comprises associating the biocompatible prosthetic valve with a catheter.

In at least one embodiment, a prosthetic trans-catheter deliverable valve is provided that does not include one or more biasing members within the inner flow channel of the valve. That is, with the exception of the membrane during closure of the valve (when the flow cycle is not antegrade from proximal to distal through the valve), the inner flow channel is devoid of flow channel obstructions.

In at least one embodiment, a prosthetic trans-catheter valve includes a flat membrane sheet interconnected to a frame. In at least one embodiment, a flat membrane sheet is interconnected to the abluminal surface of a frame using a plurality of sutures, wherein at least some of the sutures are applied in a buttonhole suture pattern.

Various components are referred to herein as “operably associated.” As used herein, “operably associated” refers to components that are linked together in operable fashion, and encompasses embodiments in which components are linked directly, as well as embodiments in which additional components are placed between the two linked components.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

As used herein, “sometime” means at some indefinite or indeterminate point of time. So for example, as used herein, “sometime after” means following, whether immediately following or at some indefinite or indeterminate point of time following the prior act.

Various embodiments of the present inventions are set forth in the attached figures and in the Detailed Description as provided herein and as embodied by the claims. It should be understood, however, that this Summary does not contain all of the aspects and embodiments of the one or more present inventions, is not meant to be limiting or restrictive in any manner, and that the invention(s) as disclosed herein is/are understood by those of ordinary skill in the art to encompass obvious improvements and modifications thereto.

Additional advantages of the present invention will become readily apparent from the following discussion, particularly when taken together with the accompanying drawings.

### BRIEF DESCRIPTION OF THE DRAWINGS

To further clarify the above and other advantages of various embodiments and features of the one or more present inventions, a more particular description of the one or more present inventions is rendered by reference to specific embodiments thereof which are illustrated in the appended drawings. It should be appreciated that these drawings depict only typical embodiments of the one or more present inventions and are therefore not to be considered limiting in scope. The one or more present inventions are described and explained with additional specificity and detail through the use of the accompanying drawings in which:

FIG. 1A is a side perspective view of an embodiment of a percutaneously deliverable valve with the valve membrane illustrated in a closed position;

FIG. 1B is a side elevation view of the frame suited to balloon expansion shown in FIG. 1A;

FIG. 1C is a top plan view of the frame shown in FIG. 1B;

FIG. 1D is a side perspective view of the frame shown in FIG. 1B;

FIG. 1E is a bottom perspective view of the frame shown in FIG. 1B;

FIG. 1F is a side elevation view of the frame shown in FIG. 1B, wherein the cylindrical frame is depicted in an “unrolled” or flat projection to illustrate the geometry of the frame members;

FIG. 1G is a side elevation view of another embodiment of a frame suited to self-expansion, wherein the cylindrical frame is depicted in an “unrolled” or flat projection to illustrate the geometry of the frame members;

FIG. 1H is a side elevation view of the frame shown in FIG. 1G;

FIG. 1I is a top plan view of the frame shown in FIG. 1H;

FIG. 1J is a side perspective view of the frame shown in FIG. 1H;

FIG. 1K is a bottom perspective view of the frame shown in FIG. 1H;

FIG. 1L is a side perspective view of an embodiment of a membrane sheet and its attachment to a frame in accordance with at least one embodiment described herein;

FIG. 2 is a simplified distal end view of an embodiment of a frame illustrating relative locations of the distal ends of two distally positioned frame projections located approximately 180 degrees apart;

FIG. 3 is a simplified distal end view of an embodiment of a frame illustrating relative locations of the distal ends of four distally positioned frame projections located approximately 90 degrees apart;

FIG. 4 is a perspective view of an embodiment of a schematic of a frame having optional stabilization framework with circumferential supports;

FIG. 5 is a perspective view of an embodiment of a schematic of a frame having optional stabilization framework with radial supports;

FIG. 6 is a flow chart of a method of constructing an embodiment of a prosthetic heart valve as described herein;

FIG. 7 is flow chart of a method of deploying an embodiment of a prosthetic heart valve as described herein; and

FIG. 8 is a schematic of a heart showing an embodiment of a heart valve as described herein implanted within a heart.

The drawings are not necessarily to scale.

### DETAILED DESCRIPTION

Embodiments of the one or more inventions described herein include one or more devices, assemblies and/or methods related to prosthetic heart valves and to prosthetic blood vessel valves. A prosthetic heart valve in accordance with at least one embodiment described herein can be surgically implanted, such as by percutaneous, trans-catheter delivery, to the implantation site within the patient. One or more embodiments of the prosthetic heart valves described herein have application for at least aortic and pulmonary valve positions, including for structural defects and diseased valves. Other embodiments have application to the vascular system and in particular to the vein system. When reduced in scale they have particular application to the branch veins of the body and the extremities. The descriptions for these devices are effectively provided in the descriptions and specifications provided for the inventive percutaneously implantable heart valve device.

In at least one embodiment, biocompatible material is mounted to a frame to form an implantable prosthetic heart valve, and then at a later time, the implantable prosthetic heart valve is implanted within a patient, such as by way of a percutaneous, trans-catheter delivery mechanism. The percutaneously implantable heart valve is suitable for implantation into a native (orthotopic or ectopic) valve seat of a patient. Once implanted, the prosthetic heart valve serves to regulate the flow of blood associated with the patient's heart by allowing forward blood flow and substantially preventing backflow or valvular regurgitation.

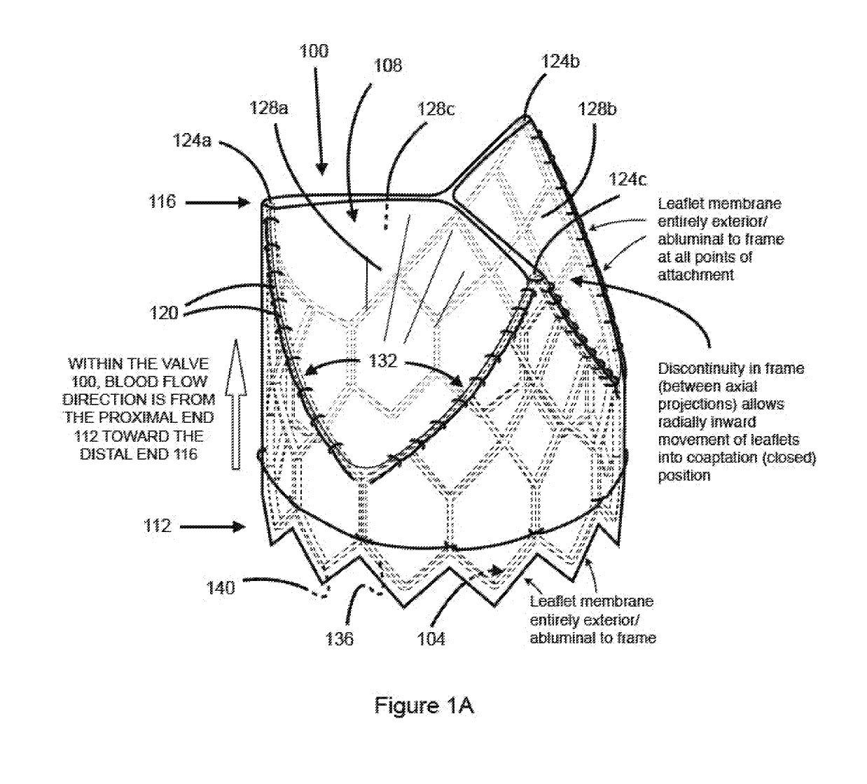

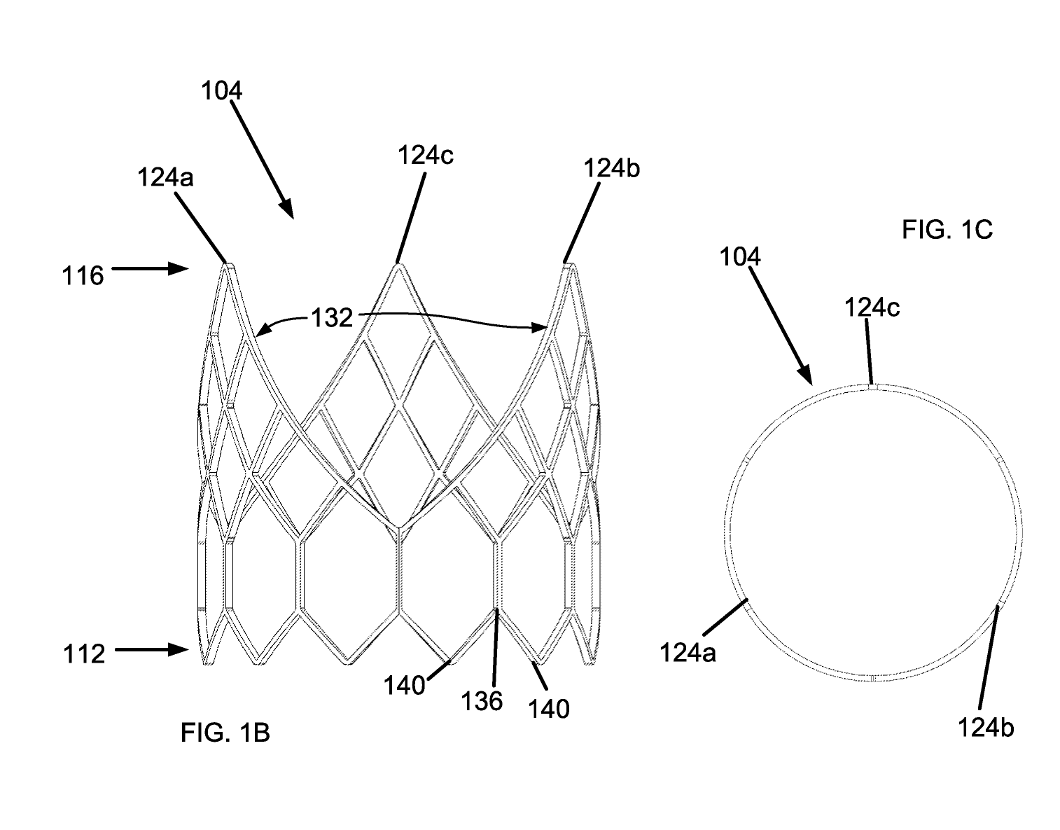

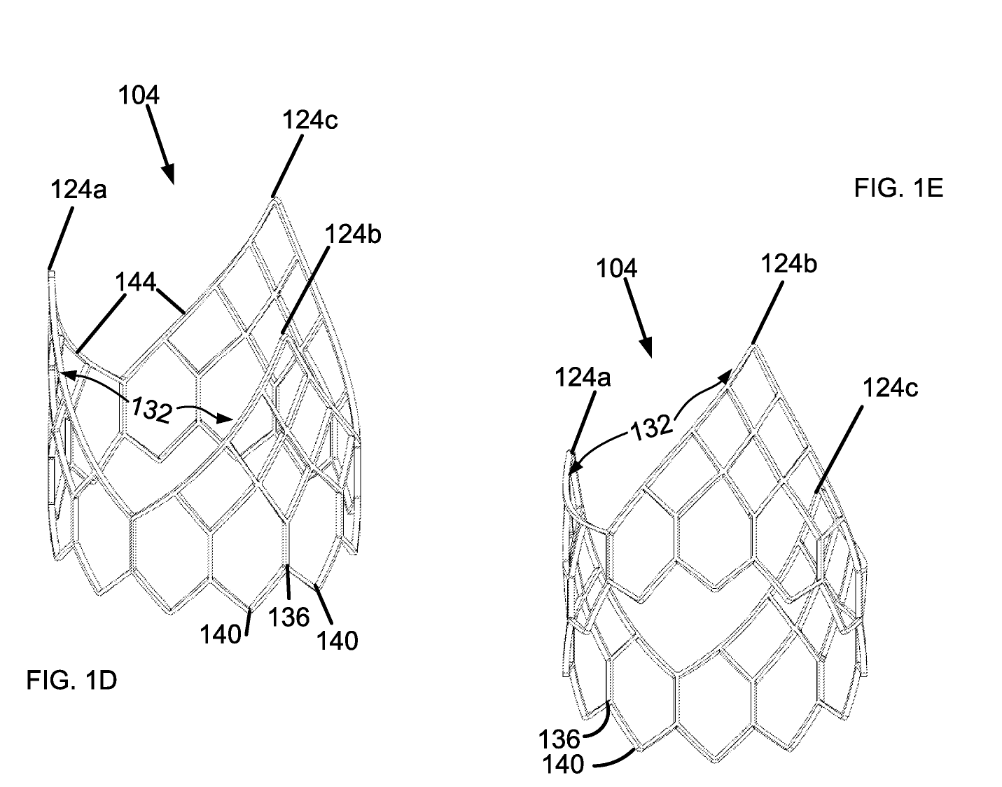

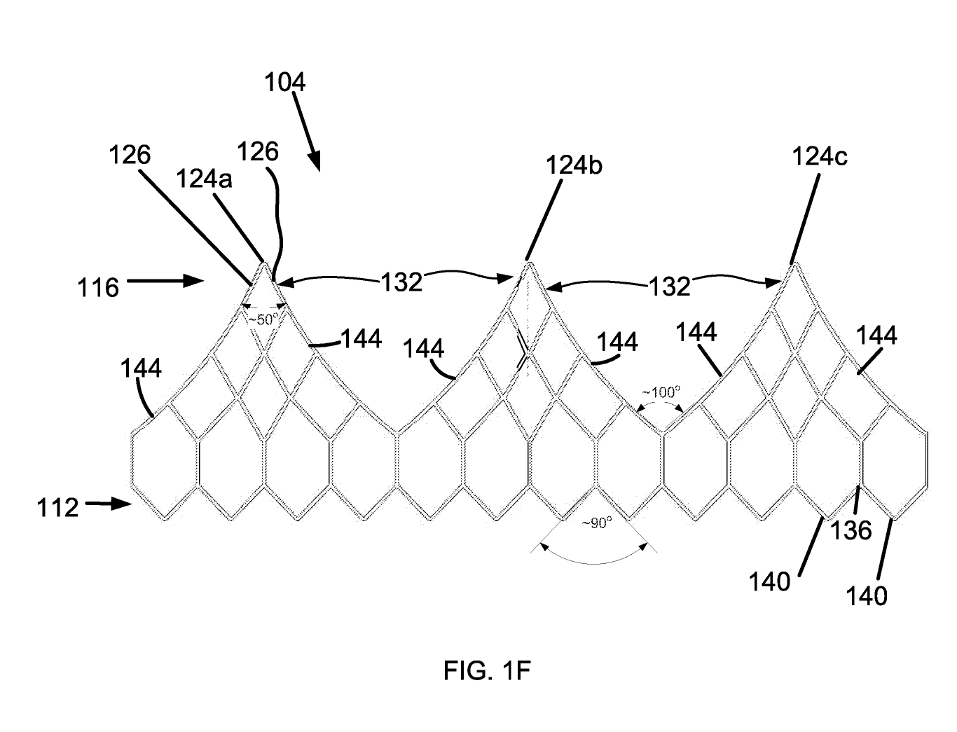

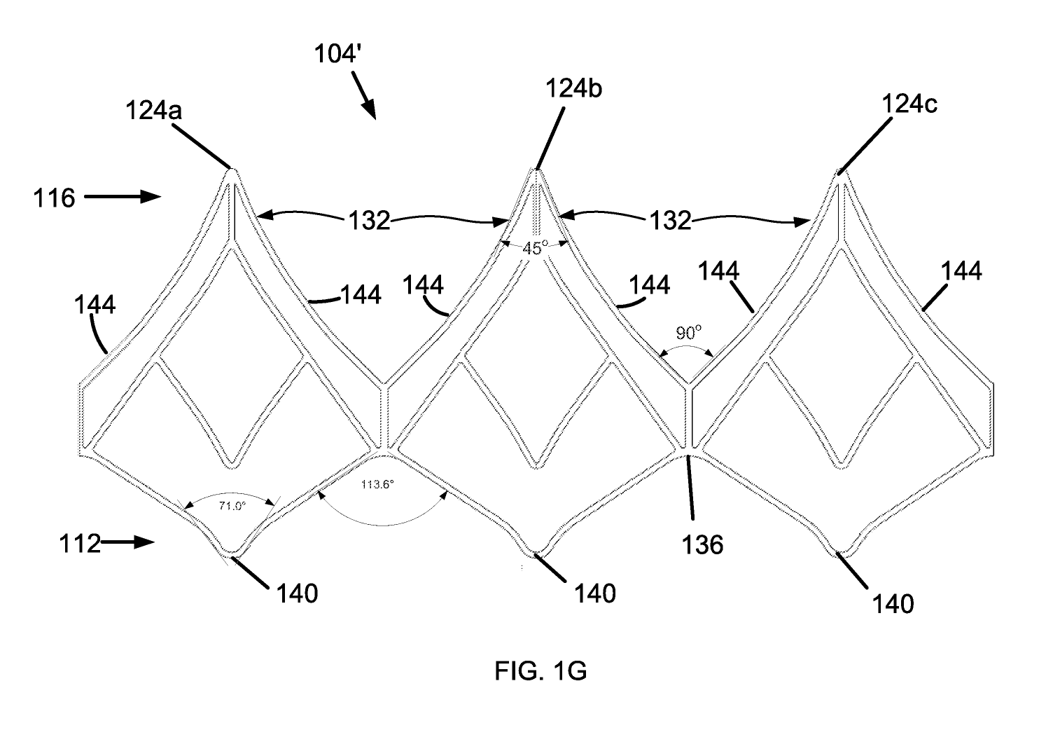

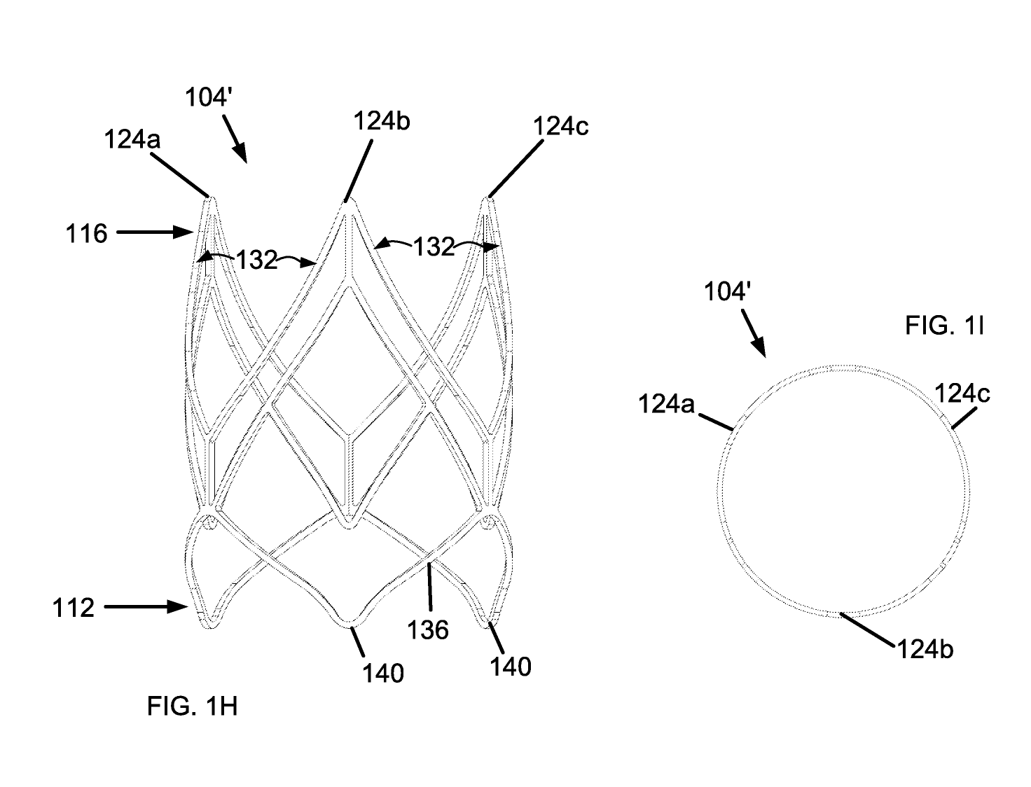

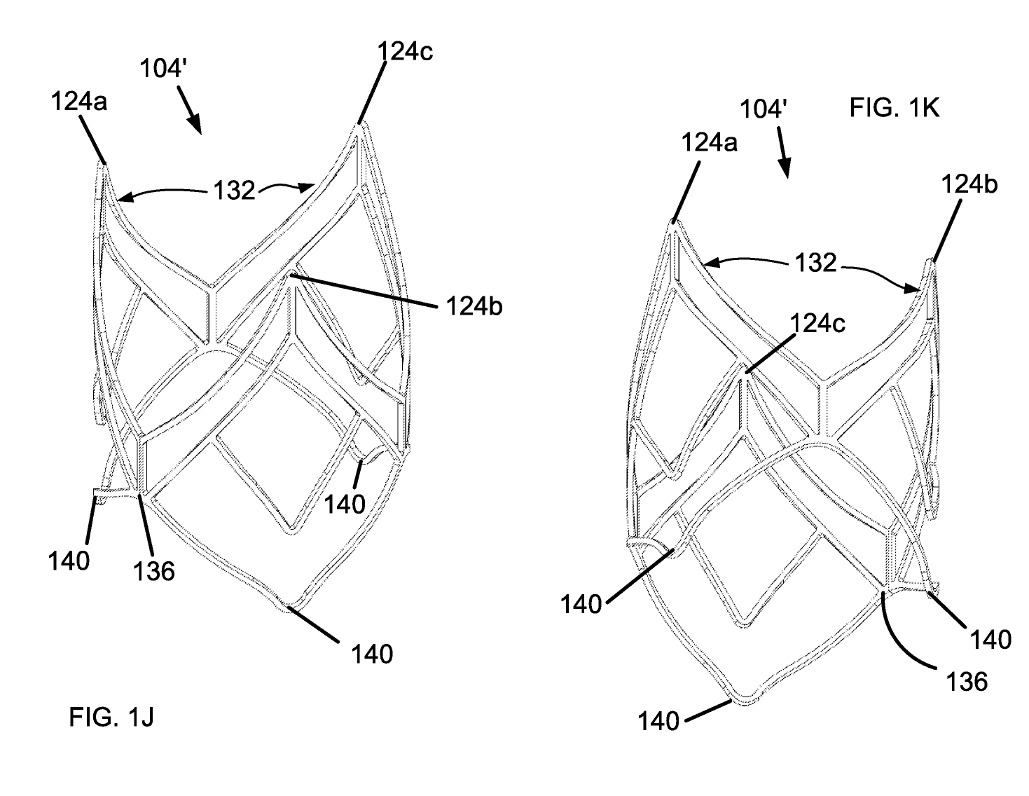

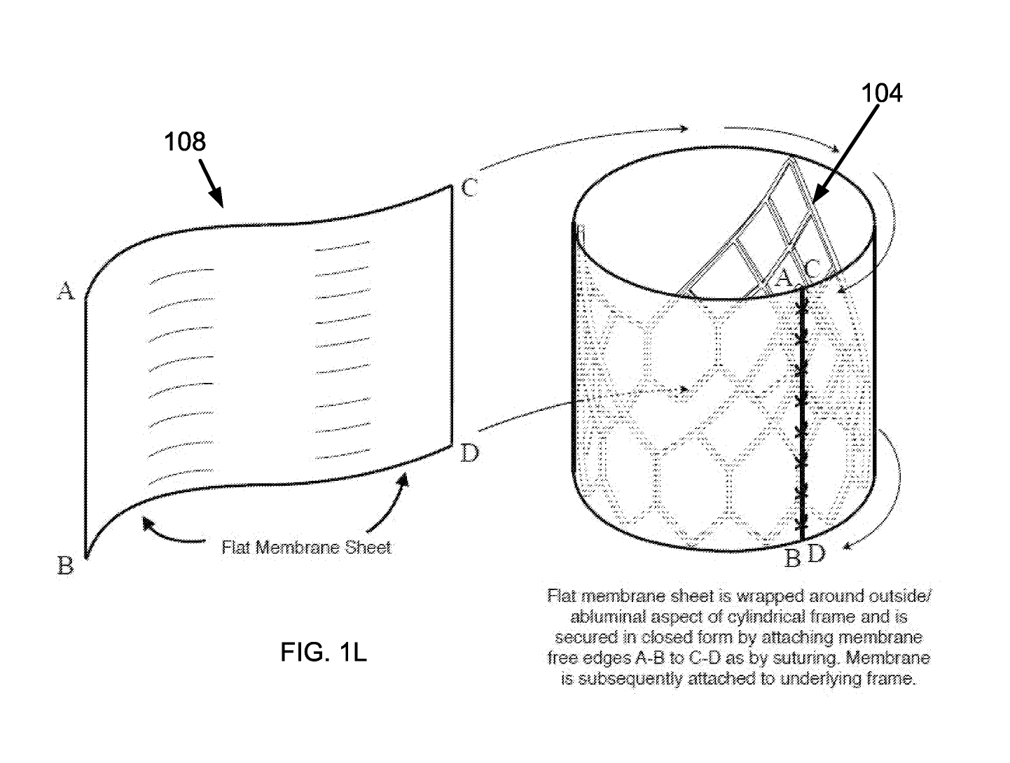

Referring now to FIG. 1A, and in accordance with at least one embodiment, an implantable prosthetic heart valve 100 is shown that includes a frame 104 and a single layer membrane sheet 108, such as a biocompatible tissue membrane sheet. All or substantially all of the membrane sheet 108 is located on the exterior or abluminal side of the frame 104 between the proximal end 112 and the distal end 116 of the frame 104 when the valve leaflets are in the operationally fully open position and in any case at all points of attachment. The implantable prosthetic heart valve 100 includes a proximal (upstream) portion/margin of membrane sheet 108 that is circumferentially attached to and residing entirely upon the abluminal surface of the frame 104. In at least one embodiment, the membrane sheet 108 is connected to the frame 104 by a plurality of sutures 120. In at least one embodiment, the plurality of sutures comprise curved lines of attachment, axially concave to the distal end 116 of the frame, along the frame members at the frame's distal edge interconnecting the distally extending frame projections 124a-c. It is to be understood that alternate ways of attaching the membrane sheet 108 to the frame 104 may be used, such as staples, an adhesive, an anchoring ring, one or more bands, clips or combinations of the foregoing.

By whatever technique of attachment, the lines of attachment by which the arcuate proximal basal margin of each leaflet is anchored to the arcuate distal edge of the frame act to distribute the force loads acting on the leaflets along these lines while in the operationally closed position. The securement of the leaflets in this manner is advantageous in a high-pressure application such as the aortic valve position. Moreover, these lines of attachment also act to seal the proximal basal margin of each cusp to the frame and are critical in the case of aortic valve implantation, because some portion of these arcuate cusp margins are likely to be disposed “above” (downstream) of the aortic valve annulus and without anatomic luminal contact to the outer aspect of the valve at this level. As such, those portions that are disposed in the “suprannular” position after implantation can be subject to high pressure blood being injected between the leaflet layer and the frame which can in turn lead to acute and chronic compromise of valve function. The specific form of leaflet attachment provided in the inventive valve addresses this problem that arises as a consequence of the abluminal/exterior position of the leaflet membrane in relation to the frame.

In at least one embodiment, the plurality of sutures 120 attaching the leaflet membrane to the distal arcuate portions of the distal edge of the frame comprise, for each arcuate segment 144, a continuous series of “buttonhole”-technique sutures 120 wherein the segments of suture interconnecting the knots are disposed to the outer/abluminal surface of the membrane. This suture configuration advantageously imposes a small biasing effect upon the leaflet towards the operationally closed position.

With regard to particular material types that may be used to form the membrane sheet, in at least one embodiment the membrane sheet 108 forming the cusp or leaflet portions includes a one-piece, single layer sheet of biocompatible membrane, such as fixed mammalian pericardium tissue or synthetic biocompatible material such as ePTFE. In at least one embodiment, the membrane sheet is made from a tissue preparation process that yields a leaflet material of suitable strength and durability for use in a prosthetic trans-catheter deliverable heart valve. The content of WO 2011/109450A2 published on Sep. 9, 2011, is incorporated herein by reference. Although not preferred, one or more embodiments may alternatively comprise a plurality of sections of membrane sheet connected to form a contiguous sheet.

In at least one embodiment, the membrane sheet is a single layer of a substantially homogenous material. In at least one embodiment, the membrane sheet is an unlaminated single layer of material. In at least one embodiment, the membrane sheet is a single layer of material that does not include any reinforcement, such as reinforcing fibers. In at least one embodiment, the membrane sheet is a single layer of treated pericardium tissue. In at least one embodiment, the membrane sheet is a single layer of a synthetic film.

The frame 104 may include a balloon expandable material. Alternatively, the frame 104 may include one or more of a self expanding alloy such as nitinol, stainless steel, cobalt chromium, bioabsorbable metal, and non-elastic bioabsorbable plastic, such as polylactides, polyglycolides, their co-polymers, or polydioxanones. As further seen in FIGS. 1A-1F, in at least one embodiment the geometry of the frame 104 at the distal end 116 may include three distally extending frame projections 124a, 124b and 124c. This configuration is described for exemplary purposes. Accordingly, alternate configurations may be used, including collapsible and expandable percutaneously deliverable frames that include two, four, five or any multiple number of distally extending frame projections, provided the configuration in combination with the abluminally situated single layer membrane sheet 108 accommodates inward closure of the membrane sheet 108 sufficiently to facilitate operational closure of the valve after being implanted. Thus, those skilled in the art will appreciate that configurations shown and described herein are for purposes of enablement, and therefore, alternate configurations from those shown are encompassed by the claims. Consistent with the foregoing, the distally extending frame projections 124a-c are spaced apart around the circumference of the frame 104 as appropriate to facilitate closure of the membrane sheet 108 when the flow cycle is not antegrade from proximal to distal through the valve.

Referring still to FIGS. 1A-1F, in at least one embodiment, the frame 104 has three distally positioned inverted “v” members also referred to herein as distally extending frame projections 124a-c located at substantially equal angular distances apart from each other at the distal end 116 of the frame 104. Alternatively, each of these distally extending frame projections may take other forms such as a single projecting beam or an extending loop formed of a continuous loop of wire. Accordingly, in at least one embodiment, each inverted “v” member or distally extending frame projection 124a-c is about 120 degrees (at the point or apex of the inverted “v” members) away on either side from the other two inverted “v” members at the distal end 116 of the frame 104. In at least one embodiment, the inverted “v” members serve as attachment locations for the membrane sheet 108. In at least one embodiment, the “v” members are integral parts of a generally arcuate configuration of frame members spanning the distal frame edge between the distally extending frame projections 124a-c such that each arcuate span forms: 1) the radially outermost margin of a leaflet cusp; and 2) the line of attachment of each leaflet membrane to the distal edge of the frame. In at least one embodiment, the proximal end 112 of the frame 104 includes a continuous framework, although minor axially oriented recessions 136 in the framework are situated between the proximal-most portions 140 of the frame 104.

With further reference to FIGS. 1B-1F, in at least one embodiment, the struts 126 forming the inverted “v” members are located between approximately 40 to 90 degrees apart, and more preferably, at between approximately 50 to 70 degrees apart. By way of example and not limitation, as shown in the example depicted in FIG. 1F, the struts 126 forming distally extending frame projection 124a are about 50 degrees apart. The angular values provided herein are given for purposes of enablement and for exemplary purposes, and are not intended to be limiting. Other values are possible, and such other values are within the scope of the one or more present inventions.

Referring again to FIG. 1A, cusp or leaflet portions 128a, 128b, and 128c reside between the spaced apart distally extending frame projections 124a-c. More particularly, circumferential discontinuities 132 in the frame 104 substantially correspond to the location of leaflet portions 128a-c in the membrane sheet 108. That is, since the membrane sheet 108 is situated exterior of the frame 104, including at the frame projections 124a-c, the absence of framework, internal struts or other types of support for a portion of the distally located membrane sheet 108 allows the abluminally positioned membrane sheet 108 to occupy an area within the flow path of the valve 100 when the flow cycle is not antegrade from proximal to distal through the valve. Therefore, when flow conditions are not antegrade, the leaflet portions 128a-c operate to close the valve 100 because of the absence of framework circumferentially between the distally extending frame projections 124a-c allows the leaflet portions 128a-c of the membrane sheet 108 to close radially inward.

Referring again to FIG. 1A, in the closed position, the leaflet portions 128a-c reside within the interior flow channel or lumen of the valve 100. Accordingly, the valve 100 includes a biocompatible membrane with a distal (downstream) portion/margin that is attached to the abluminal/exterior aspect of the frame 104 at at least two or more points (at or near the apices of the distally extending frame projections 124a-c) corresponding to two or more valve leaflet commissures, wherein the free edge of the membrane sheet 108 between the points of attachment constitutes the free edge of the valve leaflets or leaflet portions 128a-c that are free to move radially inward into a closed position contacting the other leaflet or leaflets, and radially outward into an open position.

In at least one embodiment, when the leaflets 128a-c are in their open position, the membrane sheet 108 at the distal end 116 resides entirely to the radial exterior of the frame 104 including at the distally extending frame projections 124a-c. Accordingly, when flow conditions are antegrade, the leaflets 128a-c extend radially outward from the lumen of valve 100.

In at least one embodiment, the membrane sheet 108, including the material constituting the operating leaflets portions 128a-c, is exterior/abluminal to the frame 104 and may be continuous from the leaflet portions 128a-c to the proximal end 112 of the frame 108. Alternatively, the membrane sheet 108 does not have to extend abluminally along the entire axial length of the frame 104 from the distal end 116 to the proximal end 112. More particularly, with limited proximal coverage, the membrane sheet 108 may only cover a portion of the abluminal surface of the frame 104 and reside at the distal end 116 and extend axially along the abluminal surface sufficiently to provide leaflet portions 128a-c such that there is enough membrane sheet 108 to cover the discontinuities in the frame 104 and thus function as leaflet portions 128a-c by moving radially inward and outward through the frame discontinuities 132. For such a configuration the membrane sheet 108 needs to extend proximally from the distal end 116 a sufficient proximal distance so as to provide a sufficient seal against leakage/regurgitation through the frame 104. Simply stated, the membrane sheet 108 needs to extend axially only a limited distance axially in the proximal direction, that being to slightly beyond the annular intersection or the valve seat formed between the abluminal surface of the membrane sheet 108 situated against the native tissue. Therefore, the proximal extent of the membrane tissue 108 beyond the intersection of the valve 100 against the native tissue may vary.

In at least one embodiment, the membrane sheet 108 may wrap around the proximal edge 136 of the frame 104 so as to make a continuous inner/luminal layer within the proximal end 112 of the frame 104. In contrast, leaving a portion of the proximal end 112 uncovered by the membrane sheet 108 permits the frame to provide additional structure. By way of example, the proximal end 112 can incorporate other structural elements including flared or hooked frame projections for effective securement of the implanted valve. Such configurations have applicability to providing advantageous structure for certain valve implantation sites, such as the mitral valve.

In at least one embodiment, the membrane sheet 108 may wrap around the proximal edge of the frame 104 so as to make a continuous inner/luminal layer within the proximal end 112 of the frame 104. That is, the valve 100 does not require the membrane sheet 108 to extend proximally to the proximal edge 136 of the frame 108, however, the membrane sheet 108 may extend proximally including to the proximal end 112, and indeed, the membrane sheet 108 may wrap around the proximal edge 136 to the luminal side of the frame 104.

With reference to FIG. 1F, a side elevation view of the cylindrical frame 104 is depicted in “unrolled” flat projection to illustrate the geometry of the frame members. The structural differences of the frame 104 at the proximal end 112 and distal end 116 are readily apparent, with the areas of circumferential discontinuities 132 observable between the distally extending frame projections 124a-c. Each circumferential discontinuity 132 includes a pair of generally arcuate side portions 144 that, in at least one embodiment, include a concave (in relation to the distal end of the frame) shape relative to the circumferential discontinuity 132. These arcuate spanning side portions 144 form: 1) lines of attachment of the leaflet membrane to the frame; and 2) the proximal/radially outermost margin of the leaflet cusp, along which are borne the forces exerted upon the closed leaflets. While the leaflets are attached to the arcuate side portions 144 as by suturing, the mobile leaflet portions and the cuff portion of the membrane are preferably continuous, formed of a single sheet of biocompatible membrane disposed around and upon the abluminal aspect of the frame. As noted above, to attach the single layer membrane sheet 108 to the arcuate side portions 144, sutures may be applied using a continuous series of “buttonhole”-technique sutures 120 wherein the segments of suture interconnecting the knots are disposed to the outer/abluminal surface of the membrane. This suture configuration advantageously imposes a small biasing effect upon the leaflet towards the operationally closed position.

Referring now to FIGS. 1G-1K, an alternative embodiment comprising a frame 104′ suited to self-expansion is shown is shown. When comparing frame 104 to frame 104′, differences in the frame structure are apparent. However, both frames 104 and 104′ have circumferential discontinuities 132 that substantially correspond to the location of leaflet portions 128a-c in the membrane sheet 108. Again, since the membrane sheet 108 is situated exterior of the frame 104′, including at the frame projections 124a-c, the absence of framework, internal struts or other types of support for a portion of the distally located membrane sheet 108 allows the abluminally positioned membrane sheet 108 to occupy an area within the flow path of the valve 100 when the flow cycle is not antegrade from proximal to distal through the valve. Similar to frame 104, the location of the circumferential discontinuities 132 in frame 104′ allow the leaflet portions 128a-c operate to close the valve 100 because of the absence of framework circumferentially between the distally extending frame projections 124a-c in frame 104′ allows the leaflet portions 128a-c of the membrane sheet 108 to close radially inward. Also similar to frame 104, each circumferential discontinuity 132 includes a pair of generally arcuate side portions 144 that, in at least one embodiment, include a concave (in relation to the distal end of the frame) shape relative to the circumferential discontinuity 132. These arcuate spanning side portions 144 form: 1) lines of attachment of the leaflet membrane to the frame; and 2) the proximal/radially outermost margin of the leaflet cusp, along which are born the forces exerted upon the closed leaflets.

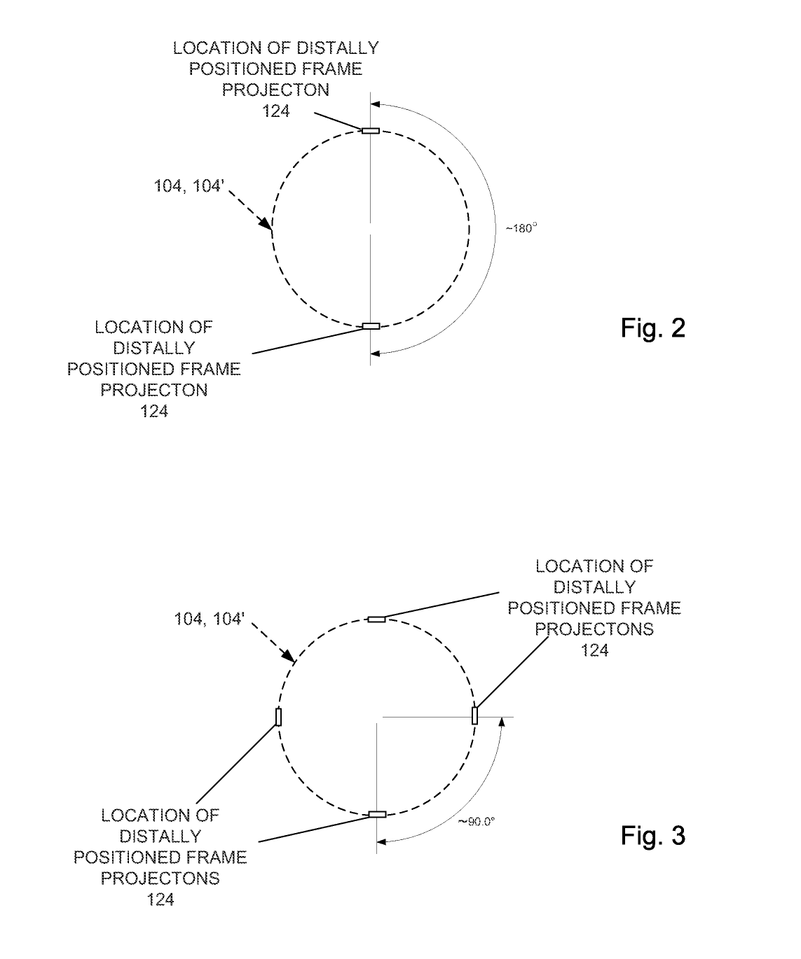

As noted above, although the embodiment shown in FIG. 1A illustrates a frame 104 including three distally extending frame projections 124a-c, an alternative number of distally extending frame projections may be used, thereby yielding an implantable prosthetic heart valve with fewer or greater than three cusps. By way of example, and with reference now to FIG. 2, for a frame having two distally extending frame projections 124 that are positioned at substantially diametrically opposite sides of the frame's circumference, then two cusps would be provided. Similarly, and with reference now to FIG. 3, for a frame having four distally extending frame projections 124 that are positioned with substantially 90 degrees of separation from one another around the frame's circumference, then four cusps would be provided.

Referring now to FIG. 1L, a frame 104 is shown relative to a single layer membrane sheet 108. The illustrated single layer membrane sheet 108 includes substantially straight edges. However, in at least one embodiment, the distal free edge of each membrane leaflet portion has a non-linear shape. Preferentially when the leaflet free edge is not linear, it is cut in the shape of a parabola with central axis of curvature aligned to the center of the free edge of the leaflet. This effectively extends the coaptation margin and area of the leaflet free edge for a given leaflet radius, reduces the pressure on the contacting leaflet areas when the valve is closed and improves the effectiveness of orifice sealing in closure. Accordingly, free edge shapes for the leaflets are cut from the corresponding edge of the flat sheet membrane before wrapping and mounting of the membrane upon the frame.

Alternatively, in at least one embodiment, the circumference of the membrane exceeds the outer circumference of the frame. The membrane is then gathered in folds or pleats and attached at the proximal (inlet) end of the frame so as to reduce the effective circumference of the membrane at the proximal end of the frame to equal that of the frame at this level. While the proximal end of the encircling membrane sheet is then directly apposed to the abluminal aspect of the frame for secure attachment, the leaflet free edge of the membrane at the distal (outlet) end of the valve remains at the original larger circumference. This has the effect of increasing the length of each leaflet free edge and the area of each leaflet for a given radius of frame, and is useful to improve valve function, especially for large valve diameters. It will be understood that various curved and polygonal membrane shapes may be used to achieve various three dimensional leaflet shapes in a similar manner. Accordingly, in at least one embodiment, a prosthetic trans-catheter deliverable valve is provided that includes a membrane sheet formed into a tubular shape, wherein a circumference of the tubular shape is greater than a circumference of a radially adjacent portion of the frame. In at least one embodiment, a circumference of the tubular shape is between about 5 to 25% greater than a circumference of a radially adjacent portion of the frame. More preferably, a circumference of the tubular shape is between about 7 to 20% greater than a circumference of a radially adjacent portion of the frame. More preferably yet, a circumference of the tubular shape is between about 10 to 15% greater than a circumference of a radially adjacent portion of the frame. The difference in the circumference of the membrane sheet as compared to the radially adjacent portion of the frame provides leaflet portions that extend within the lumen along lines of apposition with improved sealing characteristics relative to a membrane sheet having a circumference that is substantially the same as the circumference of a radially adjacent portion of the frame.

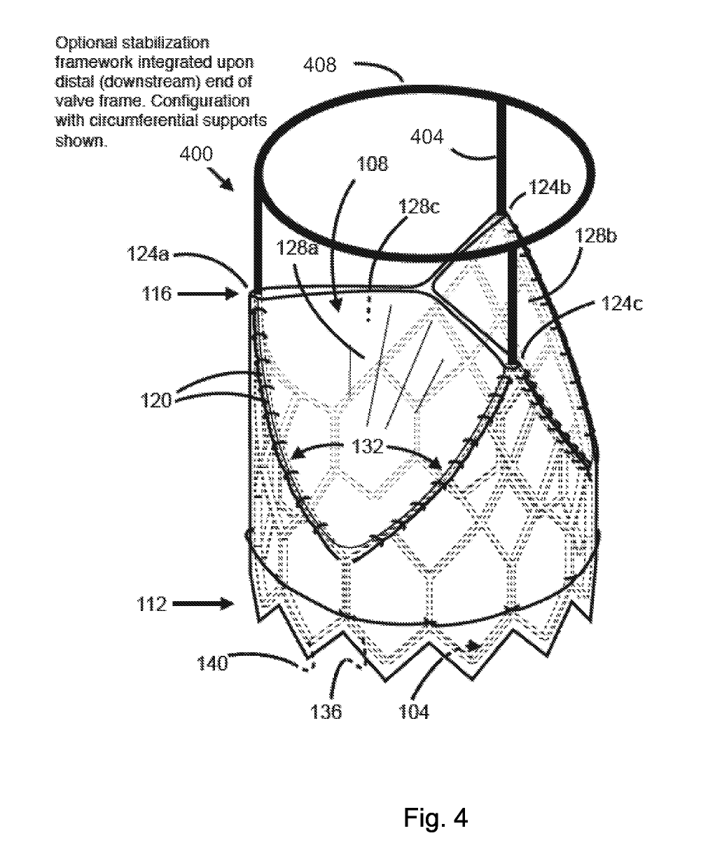

Referring now to FIG. 4, and in accordance with a separate embodiment, the frame 104 may optionally include a distally extending stabilizing framework 400 that includes axially oriented support members 404 extending from the distally extending frame projections 124a-c. In at least one embodiment, a distally-positioned circumferential ring, or alternatively, a circumferentially segmented lattice 408 interconnects the axially oriented support members 404. The stabilization framework is located distally of the membrane sheet 108 that is attached to the frame 104.

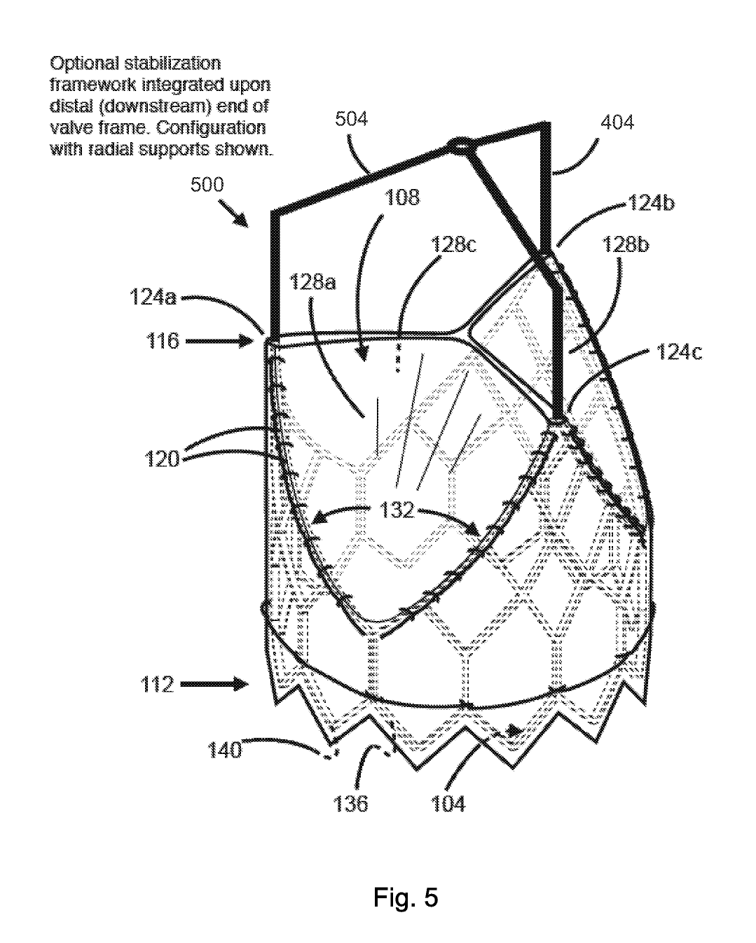

Referring now to FIG. 5, and in accordance with yet a separate embodiment, an alternative to the stabilization framework of FIG. 4 is shown. More particularly, similar to the distally extending stabilizing framework 400, distally extending stabilizing framework 500 includes a plurality of axially oriented support members 404 that extend from the distally extending frame projections 124a-c; however, a plurality of radial support members 504 are used to interconnect the axially oriented support members 404, thereby providing additional stability to the distal end 116 of the frame 104. In addition, at the central point of intersection of the radial support members, a small ring or short tube coaxially aligned with the central axis of the valve and frame may be provided in order to allow for the coaxial passage of a guide wire such that coaxial alignment of the distal support framework may be facilitated during valve deployment.

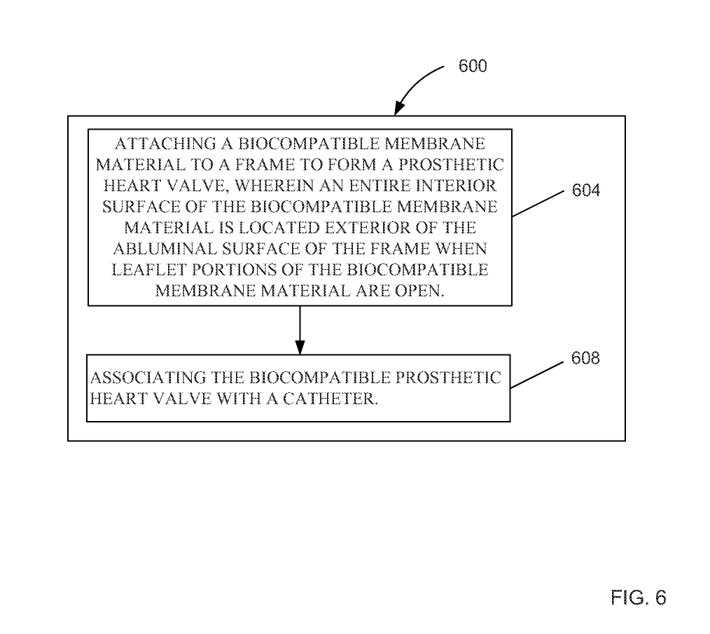

With reference now to FIG. 6, and in accordance with at least one embodiment, a method 600 of constructing a prosthetic heart valve or a prosthetic vascular valve is provided. At 604, the method includes attaching a biocompatible membrane material to a frame to form a prosthetic heart valve, wherein an entire interior surface of the biocompatible membrane material is located exterior of the abluminal surface of the frame when leaflet portions of the biocompatible membrane material are in the operationally open position. As described above, a number of different ways of attaching the membrane sheet to the frame may be used, such as by suturing the membrane sheet to the exterior of the frame. At 608, the method includes associating the biocompatible prosthetic heart valve or prosthetic vascular valve with a catheter. The 604 step of associating may be preformed at a different location than the step 608 of attaching.

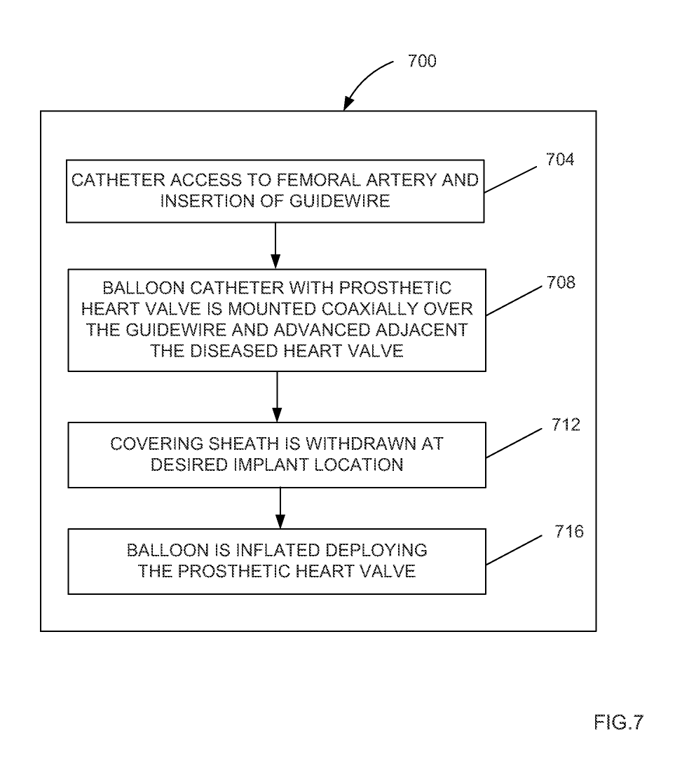

Referring now to FIG. 7, a flow chart illustrating the general procedure associated with implantation of the percutaneously deliverable heart valve 100 is provided. However, those skilled in the art will understand that with appropriate modification (e.g., changing the vascular entry location) the methodology also has application to a percutaneously deliverable blood vessel valve.

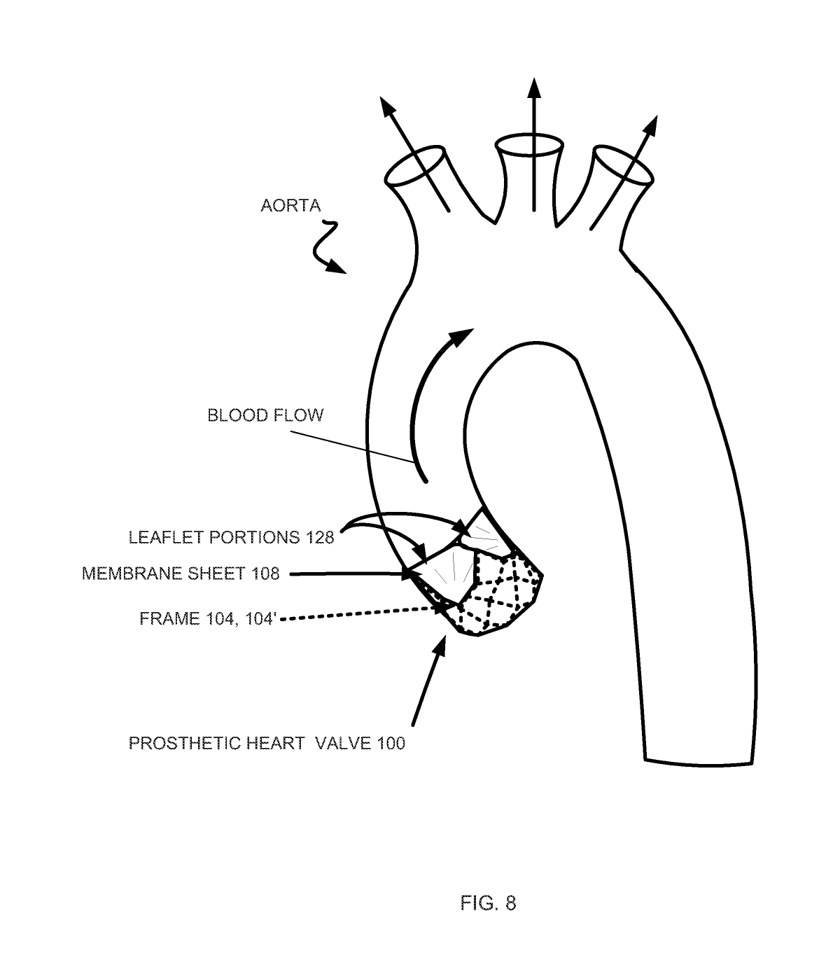

At 704, catheter access is gained to the patient's femoral artery and a guidewire is placed through the plane of the diseased valve that is targeted to receive the implant. Thereafter, the percutaneously deliverable heart valve 100 is removed from its packaging. If the valve was not mounted upon or otherwise associated with a delivery catheter at manufacture, then the valve is cleaned and rinsed and radially compressed upon the delivery catheter and constrained within a covering sheath coaxial to the delivery catheter. The prosthetic heart valve assembly, including its lumens, is preferably flushed and prepared in the usual fashion for standard balloons and catheters that do not contain a biocompatible tissue. At 708, the carrier catheter or balloon catheter is then coaxially mounted and advanced over the guidewire, such as under fluoroscopic vision initially to the level of the great vessel where it can be inspected under fluoroscopy. At 712, and after the nominal position and configuration is confirmed, the delivery system is advanced through the plane of the diseased valve under fluoroscopy, and the covering sheath is withdrawn, either at this point or during the advance prior to it, thus exposing the mounted implantable prosthetic heart valve 100 in place. At 716, in the case of a balloon expandable frame, the balloon is then inflated, deploying the percutaneously deliverable heart valve 100 in the plane of the valve. The deployed prosthetic heart valve 100 is shown in FIG. 8, wherein the percutaneously deliverable heart valve 100 serves to properly control the flow blood.

One or more of the embodiments of the percutaneously deliverable heart valve described herein may be implanted into the patient using a balloon-expandable frame or a self-expanding frame. Expandable frames are generally conveyed to the site of the target valve on balloon catheters. For insertion, the expandable frame is positioned in a compressed configuration along the delivery device, for example crimped onto the balloon of a balloon catheter that is part of the delivery device intended for coaxial mounting on a guidewire. After the expandable frame is positioned across the plane of the valve, the expandable frame is expanded by the delivery device. For a self-expanding frame, commonly a sheath is retracted, allowing expansion of the self-expanding frame.

In at least one embodiment, the frame comprises a metal alloy frame possessing a high strain design tolerance that is compressible to a relatively small diameter. By providing a device with a low profile, the implantable prosthetic heart valve allows standard retrograde arterial aortic delivery via femoral artery insertion, without surgical cutdown or general anesthesia.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

The one or more present inventions, in various embodiments, include components, methods, processes, systems and/or apparatus substantially as depicted and described herein, including various embodiments, subcombinations, and subsets thereof. Those of skill in the art will understand how to make and use the present invention after understanding the present disclosure.

The present invention, in various embodiments, includes providing devices and processes in the absence of items not depicted and/or described herein or in various embodiments hereof, including in the absence of such items as may have been used in previous devices or processes (e.g., for improving performance, achieving ease and/or reducing cost of implementation).

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

Moreover, though the description of the invention has included description of one or more embodiments and certain variations and modifications, other variations and modifications are within the scope of the invention (e.g., as may be within the skill and knowledge of those in the art, after understanding the present disclosure). It is intended to obtain rights which include alternative embodiments to the extent permitted, including alternate, interchangeable and/or equivalent structures, functions, ranges or acts to those claimed, whether or not such alternate, interchangeable and/or equivalent structures, functions, ranges or acts are disclosed herein, and without intending to publicly dedicate any patentable subject matter.

What is claimed is:

1. A percutaneous, trans-catheter prosthetic valve for implantation in a patient, comprising:
a frame including an abluminal surface extending between a proximal end of the frame and a distal end of the frame, wherein the frame is collapsible and expandable and adapted for trans-catheter delivery; and
a biocompatible tissue material mounted to the abluminal surface of the frame to form a plurality of valve leaflets, wherein an entire interior surface of the biocompatible tissue material between the proximal end of the frame and the distal end of the frame resides radially exterior to the abluminal surface of the frame:
(a) at all points of attachment; and
(b) when the plurality of valve leaflets are in an operationally fully open position.

2. The percutaneous, trans-catheter prosthetic valve of claim 1, wherein the frame comprises a metal alloy substantially configured as tubular stent member.

3. The percutaneous, trans-catheter prosthetic valve of claim 2, wherein a proximal portion of the frame includes a ring.

4. The percutaneous, trans-catheter prosthetic valve of claim 2, wherein a proximal portion of the frame comprises a circumferential zig-zag of wire.

5. The percutaneous, trans-catheter prosthetic valve of claim 2, wherein a proximal portion of the frame includes a lattice.

6. The percutaneous, trans-catheter prosthetic valve of claim 5, wherein the lattice is circumferentially continuous.

7. The percutaneous, trans-catheter prosthetic valve of claim 5, wherein the lattice is circumferentially discontinuous.

8. The percutaneous, trans-catheter prosthetic valve of claim 1, wherein a distal end of the frame includes two or more areas of axial continuity with the proximal end, and wherein the two or more areas of axial continuity comprise axially oriented projections.

9. The percutaneous, trans-catheter prosthetic valve of claim 8, further comprising a distally positioned stabilization framework comprising at least one of circumferential or radial continuity with the axially oriented projections.

10. The percutaneous, trans-catheter prosthetic valve of claim 8, wherein the frame includes two or more regions of circumferential discontinuity through which the plurality of valve leaflets of the biocompatible tissue material move radially inward and outward in closing and opening operation, respectively.

11. The percutaneous, trans-catheter prosthetic valve of claim 1, wherein the biocompatible tissue material between the proximal end of the frame and the distal end of the frame resides substantially adjacent the abluminal surface of the frame.

12. The percutaneous, trans-catheter prosthetic valve of claim 1, wherein the biocompatible tissue material does not contact a luminal surface of the frame.

13. The percutaneous, trans-catheter prosthetic valve of claim 1, wherein an exterior surface of the biocompatible tissue material does not contact a luminal surface of the frame.

14. A prosthetic valve for implantation in a patient, comprising:
a frame including an abluminal surface extending between a proximal edge of the frame and a distal edge of the frame, the distal edge undulating axially to define at least two areas of circumferential discontinuity in the frame, wherein the frame is collapsible and expandable and adapted for trans-catheter delivery; and
a single layer of a biocompatible membrane material mounted to the abluminal surface of the frame to form leaflet portions, wherein the leaflet portions are collocated with the at least two areas of circumferential discontinuity in the frame.

15. The prosthetic valve of claim 14, wherein no portion of the biocompatible membrane material is mounted to an interior surface of the frame.

16. The prosthetic valve of claim 14, wherein the frame comprises a metal alloy substantially configured as tubular stent member.

17. The prosthetic valve of claim 16, wherein a proximal portion of the frame includes a lattice to which the biocompatible membrane material is circumferentially mounted entirely upon the abluminal surface of the tubular stent member.

18. The prosthetic valve of claim 17, wherein the lattice is circumferentially continuous.

19. The prosthetic valve of claim 17, wherein the lattice is circumferentially discontinuous.

20. The prosthetic valve of claim 14, wherein a proximal portion of the frame comprises a circumferential zig-zag of wire.

21. The prosthetic valve of claim 14, wherein the biocompatible membrane material extends between the proximal edge and the distal edge of the frame.

22. The prosthetic valve of claim 14, wherein at least some proximal portion of the frame does not include biocompatible membrane material mounted to its luminal or abluminal surfaces.

23. The prosthetic valve of claim 14, wherein a distal portion of the frame further includes a distally extending stabilizing framework comprising a plurality of axially oriented support members that each extend from a distally extending frame projection situated adjacent the at least two areas of circumferential discontinuity in the frame.

24. The prosthetic valve of claim 23, further comprising a plurality of radial support members interconnecting the plurality of axially oriented support members.

25. The prosthetic valve of claim 24, further comprising a wire guide, wherein the wire guide is coaxially aligned with an axis of the prosthetic valve, and wherein the wire guide is configured to allow for a coaxial passage of a guide wire such that coaxial alignment of the distally extending stabilizing framework may be facilitated during valve deployment.

26. The prosthetic valve of claim 25, wherein the wire guide comprises at least one of a ring and a tube.

27. The prosthetic valve of claim 14, wherein a circumference of the biocompatible membrane material is between about 5 to 25% greater than a circumference of a radially adjacent portion of the frame.

28. A method of preparing a percutaneous, trans-catheter prosthetic valve, comprising:
mounting a single layer of a biocompatible tissue material to an abluminal surface of a trans-catheter deliverable frame such that an interior surface of the biocompatible tissue material between a proximal end of the trans-catheter deliverable frame and a distal end of the trans-catheter deliverable frame resides radially exterior to and substantially adjacent the abluminal surface of the trans-catheter deliverable frame at all points of attachment and in entirety when a plurality of leaflets of the biocompatible tissue material are in a fully open position.

29. The method of preparing a percutaneous, trans-catheter prosthetic valve of claim 28, further comprising compressing and crimping the trans-catheter deliverable frame, with the biocompatible tissue material mounted thereto, upon a delivery catheter.

30. The method of preparing a percutaneous, trans-catheter prosthetic valve of claim 29, further comprising implanting the trans-catheter deliverable frame with the biocompatible tissue material mounted thereto into a patient.

31. The method of preparing a percutaneous, trans-catheter prosthetic valve of claim 28, wherein the trans-catheter deliverable frame comprises a stent.

32. The method of preparing a percutaneous, trans-catheter prosthetic valve of claim 28, further comprising mounting the trans-catheter deliverable frame and the biocompatible tissue material mounted thereto on a mandrel.

33. A method, comprising:
attaching a biocompatible membrane material to a collapsible and expandable frame to form a trans-catheter deliverable prosthetic valve, wherein an entire interior surface of the biocompatible membrane material is located exterior of an abluminal surface of the collapsible and expandable frame when leaflet portions of the biocompatible membrane material are in a fully open position.

34. The method of claim 33, wherein the attaching includes suturing the biocompatible membrane material to a distal edge of the collapsible and expandable frame that undulates in an axial direction around the collapsible and expandable frame.

35. The method of claim 33, further comprising associating the trans-catheter deliverable prosthetic valve with a catheter.
